(19) United States
(12) Patent Application Publication
(10) Pub. No.: US 2017/0362556 A1
(43) Pub. Date: Dec. 21, 2017

(54) Systems and Methods for Aseptic Sampling

(71) Applicant: GE HEALTHCARE BIO-SCIENCES CORP., MARLBOROUGH, MA (US)

(72) Inventor: Yasser Ali, Westborough, MA (US)

(21) Appl. No.: 15/532,594

(22) PCT Filed: Dec. 4, 2015
(86) PCT No.: PCT/EP2015/078643

§ 371 Date: Jun. 2, 2017

Related U.S. Application Data
(60) Provisional application No. 62/089,517, filed on Dec. 9, 2014.

(51) Int. Cl. C12M 1/00; C12M 1/12; C12M 1/32; C12M 1/34; B01L 3/00

(52) U.S. Cl. CPC C12M 23/40; C12M 37/00; C12M 33/06; B01L 3/563; C12M 41/00

(57) ABSTRACT

A product and method for sampling cells from a bioreactor, for the purposes of determine the cell count or to remove a sample and retain sterility of the sample for quality control (OC) assessment. More specifically, a product and a method for sampling of cells during a cell expansion in a bioreactor using vacuum tubes.

### FIELD OF THE INVENTION

The present invention relates to a method for sampling cells from a cell culture in a bioreactor for the purposes of, for example, determining the cell count, providing a sample for off-line analyses or removing a sample and retain sterility of the sample for quality control (QC) assessment. More particularly, the invention relates to a method for aseptic sampling of cells at one or more instances in time during a cell expansion in a bioreactor using vacuum tubes.

### DESCRIPTION OF RELATED ART

At present advanced cell therapy products are grown in a number of bioreactors that differ in their design and structure. In particular, immunotherapy applications require the rapid expansion of T lymphocytes, either nave cells or engineered cells that express a receptor to tumour cell antigen. Over the course of the cell expansion the operator may wish to understand what is happening with the cell culture environment, supply of nutrients, accumulation of waste metabolites or to remove a cell sample for further analysis.

Flexible cell culture bags are currently used to culture and expand primary peripheral blood mononuclear cells (particularly T cells) for transplantation into patients. Cells are grown within a contained cell bag and high cell densities are achieved by using media perfusion, where fresh media is added to the culture and spent media is removed. The rate of media perfusion is dependent upon the concentration of cells within the cell bag, the perfusion rate increasing with increased cell concentration. Monitoring of the growth rate and concentration of the cultured cells requires sampling from the cell bag, and generally at more than one timepoint.

The preferred prior art method for sampling is to connect a syringe to a needless port of the bioreactor and remove a sample of the cell suspension from the bioreactor. Use of a syringe to sample from the disposable bioreactor requires careful and detailed procedures to ensure sterility is maintained; a syringe is a two-way device and consequently has the potential to allow the operator to push air into the bioreactor and risk contaminating the culture. Disconnection of the syringe from the bioreactor exposes the contents of the syringe to the local environment and increases the risk of microbial contamination.

In a second preferred prior art method the user may fit a length of sterile tube connected to a 3-way valve. A syringe is fitted to the valve and used to draw sample into the tubing. The tubing is then clamped and sealed to provide a sterile cell sample. However, in this method the operator has multiple steps to complete to obtain the final sample.

When opened, the sampling port exposes the culture to the external environment which carries the risk of contamination of the culture, and each sampling instance requires drawing a portion of the sample from the cell bag. Different tubes are attached to ports on the cell bag or are passed through the ports at different instances in time for different sampling instances. Any leakage or contamination in the tubing or in the connection between the culture vessel and the tubing may introduce contamination in the cell bag. Every sampling instance is accompanied by a user attaching some sort of tubing either directly or indirectly to the cell bag, thereby increasing the risk of contamination of the cell culture. In addition, there is a likelihood of a portion of the sample being left in the tubing after the sampling instance. This residual sample may then be inadvertently carried over to the next sampling instance, thereby jeopardizing the purity of the sample obtained in the next sampling instance. Also, each sampling instance increases the likelihood of contamination of the cell culture. Hence, it is desirable to ensure that sampling is carried out in a manner which avoids introduction of contaminants into the pre-established sterile system. Furthermore, using current methods to carry out sampling from a bioreactor such as the WAVE or Xuri™ (GE Healthcare) bioreactors is often cumbersome and can leave the isolated sample exposed to the atmosphere. Sampling the bioreactor using known approaches therefore also runs the risk of providing a non-sterile sample that may fail the sterility QC check.

Consequently, in addition to the complex nature and risk of contamination associated with known sampling techniques, there also may exist an inherent limitation on the number or frequency of samplings which may be accommodated, either by reason of a limited number of sterilizable sequences to which a particular connector can be subjected to before severe degradation occurs or simply by reason of the long time needed to perform a sample withdrawal. These limitations may pose significant problems in situations where rapid and frequent sampling is required in order to monitor a potentially fast-changing situation. Still further, of course, elaborate and/or time-consuming sampling techniques can add significantly to the overall cost of the culture process.

It would therefore be desirable to have a simple and robust cell sampling method which provides a sterile sample and which exposes the cell culture to a minimal contamination risk.

### SUMMARY OF THE INVENTION

In one aspect the present invention provides a sampling device comprising:
a first conduit (1) comprising a first port (2) and a second port (3), wherein said first port (2) is configured to be fluidly connected to a bioreactor and wherein said second port (3) is fluidly connected to a venting device (4);
a plurality of sub-conduits (5) having corresponding sub-ports (6), wherein each of the plurality of sub-conduits (5) is fluidly connected to the first conduit (1) at respective connector junctions (7), and wherein each of the sub-ports (6) is fluidly connected to the first conduit (1) and configured to be operatively coupled to a vacuum tube (9);
a plurality of flow controllers (8) disposed along said first conduit (1) between each of said connector junctions (7) and disposed along each of said sub-conduits (5) between said connector junction (7) and said sub-port (6);
a plurality of vacuum tubes (9) each configured to be operatively coupled to a respective sub-port (6) of a corresponding sub-conduit (5).

In another aspect the present invention provides a method comprising sampling cells from a bioreactor into a vacuum tube (9) wherein said method comprises:
providing a sampling device as defined herein wherein said plurality of flow controllers (8) are in a closed position;
aseptically connecting said sampling device to said bioreactor;
creating a closed fluid path from said bioreactor to a sub-port (6) of said sampling device by opening a defined selection of said plurality flow controllers (8); and,
aseptically connecting said vacuum tube (9) to said sub-port (6) so that said cells to move from the bioreactor into said vacuum tube (9).

The present invention provides a method for rapid cell sampling which will maintain a sterile sample, reduce handling time and effort compared to prior art. The method of the invention involves use of a simple disposable system that in its single sampling embodiment removes the need to flush and residual cell suspension from the sampling line prior to taking a sample, which is often considered a concern when a multi-sampling device has been assembled. The method of the invention in another embodiment permits multi-sampling where the sampling device of the invention is used, which provides advantages over prior art multi-sampling methods and systems. The method of the invention is a simple, low risk and effective procedure to collect one or more samples from any bioreactor fitted with a needless port.

An advantage of the invention is that the sampling method may be used for any type of cultivated cells even very sensitive cells such as human peripheral blood mononuclear cells (PBMCs) and lymphocytes.

### BRIEF DESCRIPTION OF THE FIGURES

FIG. 1 illustrates an exemplary sampling device of the invention, which can be used to carry out the method of the invention for a plurality of sampling instances.

FIG. 2 is a flow diagram describing how to carry out a sampling instance using the sampling device illustrated in FIG. 1.

FIG. 3A shows a schematic view of an access device with a sleeved hollow needle and a connection/fitting for connection to a sampling port on a bioreactor: and

FIG. 3B shows a schematic view of the access device coupled to a vacuum container, preferably a vacuum tube. This assembly is created after the sleeved needle has been connected to the bioreactor, whereupon the vacuum tube draws the sample form the bioreactor in a unidirectional flow from the bioreactor into the tube.

### DETAILED DESCRIPTION OF THE PREFERRED EMBODIMENTS

To more clearly and concisely describe and point out the subject matter of the claimed invention, definitions are provided hereinbelow for specific terms used throughout the present specification and claims. Any exemplification of specific terms herein should be considered as a non-limiting example.

The term “sampling device” is taken to mean a device suitable for taking one or more samples from a bioreactor in an aseptic manner.

The term “conduit” generally refers to tubing suitable for the creation of a closed fluid pathway that permits the passage of material from a bioreactor to a vacuum tube. Suitable conduits include tubing made from flexible liquid-tight tubing. In one embodiment of the invention said first conduit is a continuous conduit.

The term “port” refers to an opening allowing the passage of a fluid therethrough. In the context of the device of the invention a port permits the passage of fluid either into or out of a particular feature of the device and into or out of another feature of the device.

The term “fluidly connected” means that fluid can freely pass from one feature of the device to another feature of the device.

The term “venting device” refers to any device configured to facilitate purging of a portion of a first conduit of the sampling device. Non-limiting examples of suitable venting devices are: a mechanical pump, a motorized pump, a venting valve in conjunction with a vacuum tube. In one embodiment, the mechanical pump may include a resilient container (e.g., a resilient bulb container) with at least two flow regulators or a piston based structure (e.g., a syringe) with at least one flow regulator. In one embodiment of the invention, an air filter is operatively coupled to the venting device.

The term “bioreactor” refers to any manufactured or engineered device or system that supports a biologically active environment. A bioreactor in the context of the present invention is a device or system in which cells or tissues are grown in the context of cell culture. The bioreactor may be a rigid reusable vessel made e.g. from stainless steel or a flexible cell bag designed for a single use before being discarded.

The term “sub-conduit” refers to a conduit branching off the first conduit and is typically a shorter conduit than the first conduit.

The term “sub-ports” simply means a port as defined hereinabove present on a sub-conduit.

The term “connector junction” refers to each point where the first conduit meets a sub-conduit. It is necessary that this junction is enclosed to form a liquid-tight seal in order to maintain a sterile closed system within the sampling device. This may be achieved either by virtue of the first conduit being continuous with each of the sub-conduits or by means of a connector device.

The term “configured to be coupled” means comprising features that will permit a liquid-tight seal to be formed with another named feature. For example, the feature so-configured may comprise a male Luer-Lok™ fitting and the feature to which it is configured to be coupled would therefore comprise the respective female fitting.

The term “flow controller” refers to a device that can variously permit and prevent liquid flow through the particular conduit or sub-conduit with which it is associated. A suitable flow controller has an open and a closed position to permit and prevent flow, respectively. In one embodiment the flow controller is a clamp such as a tubing clamp, many embodiments suitable for application in the device of the present invention of which are well known.

The term “disposed along” in the context of a flow controller in the present invention means that the flow controller is in place along the tubing in such a way as it can perform its function.

The term “configured to be operatively coupled” refers to the property of an article whereby it may carry out its intended function when it is associated with another article.

A “container” in the context of the present invention is meant a sterile glass or plastic container with a closure that is evacuated to create a vacuum inside the container facilitating the draw of a predetermined volume of liquid therein. In one embodiment of the invention the container is a vacuum tube. In one embodiment each sub-port comprises a needle-provided access device wherein a fluid path is created through the needle of the access device. A fluid path passage is thereby created through the needle of the access device so that fluid can flow from the bioreactor into the vacuum tube. A well-known use of such vacuum tubes is for drawing blood samples directly from a vein. A non-limiting example of such a vacuum tube is a Vacutainer™ tube. Containers under vacuum, such as the Vacutainer™ are well-established products used at hospitals and care centres for rapid blood sampling. The containers comprise a vacuum tube that aseptically draws blood through a sleeved covered needle. The vacuum tube is available in various volumes and often pre-coated with various compounds to prevent for example blood clotting. The invention exploits the features of the vacuumised containers and associated sleeved needle for use as a cell sampling device that connects to a bioreactor for cell culture through a one way needless port. The access device is preferably a sterile, single use product and offers a simplified process for the operator to remove a sample for analysis. The design of the access device with vacuum tube and sleeved needle is such that once drawn the sample will retain its sterility for further analysis.

In one embodiment of the invention the vacuum tube may be empty, in another embodiment it may be filled with selected reagents, such as cell viability stains, for example DRAQS, Hoechst, or propidium iodide. In a further embodiment the vacuum tube contains single antibodies, or multiple antibodies, such as anti-CD3, anti-CD4 or anti-CD8.

In one embodiment of the method of the invention the cells are instantly mixed with the reagent and then the tube is removed from the bioreactor and the cells are analysed, for example in respect of viable cell number or the presence of antigen.

The term “vacuum force” refers to the force that is created by virtue of the pressure differential between the bioreactor and the evacuated container that enables the passage of a sample from the bioreactor to the evacuated container.

The term “closed fluid path” means a continuous path within the sampling device of the invention through which fluid can easily flow and in which sterility can be maintained.

In one embodiment of the method of the present invention said sampling comprises a plurality of sampling instances, wherein the method of the invention as defined herein is carried out a plurality of times, each time using a different sub-conduit and sub-port of the sampling device of the invention. Purging of the sampling device is carried out in between sampling instances and following each sampling instance the relevant sub-conduit is sealed, e.g. by heat sealing, and cut to aseptically release the vacuum tube. In one embodiment, sealing takes place as close as possible to the fluid conduit of the sampling device.

The present invention will now be described in relation to a non-limiting example and the accompanying figures.

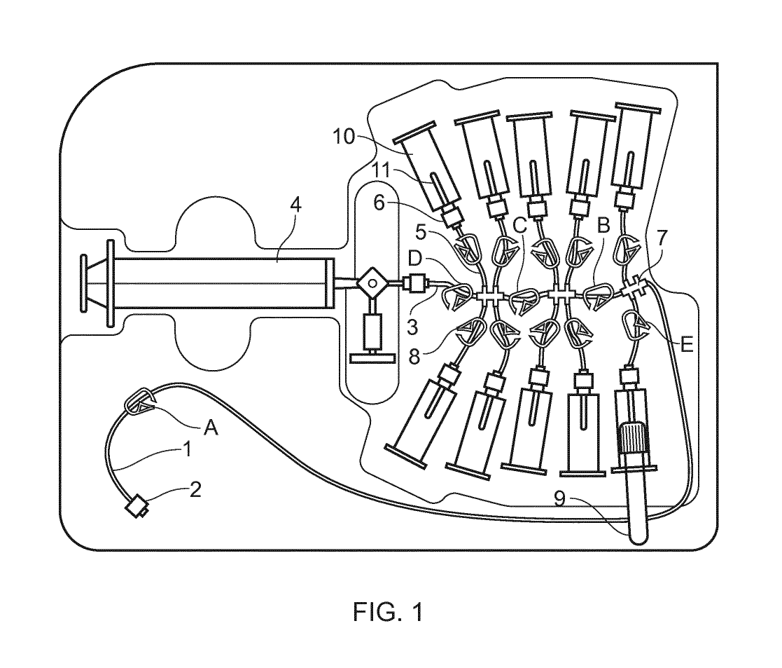

FIG. 1 illustrates an exemplary sampling device of the present invention configured to carry out a plurality of sampling instances. A first fluid conduit (1) made from flexible polymeric tubing defines a fluid passageway from a first port (2) configured to be fluidly connected to a bioreactor to the venting device (4) which is in this embodiment a syringe connected via a Luer connection. Branching off the first conduit (1) are a number of sub-conduits (5), only one of which is labelled in FIG. 1, each having a corresponding sub-port (6). A closed fluid pathway is maintained at the junction of the first conduit (1) and each sub-conduit (5) by means of rigid plastic connector junctions (7). Various small plastic tubing clamps (A-E and others that are unlabelled in FIG. 1) are positioned so that when selected clamps are open either aseptic sampling into a particular vacuum tube (9), or purging of the first conduit (1) can take place.

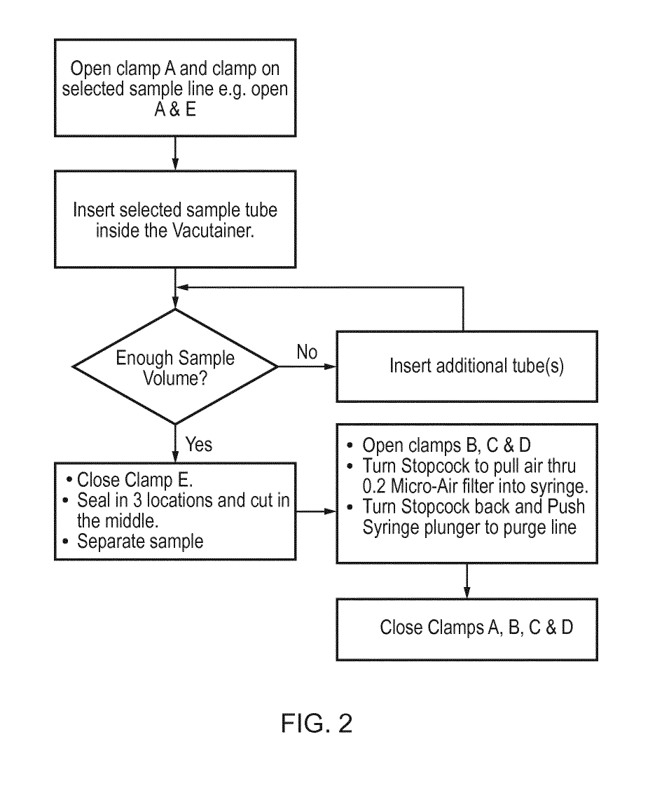

FIG. 2 presents a flow diagram showing how a sampling instance can be carried out in the sampling device illustrated in FIG. 1 when connected to a bioreactor containing e.g. a cell culture. Starting with all the clamps closed, clamps A and E are opened before inserting a vacuum tube (9) to collect a sample from the bioreactor into the tube (9). The clamps A and E need to be open before the needle will pierce the vacuum tube rubber cover to activate the negative pressure inside the sampling tube and withdraw liquid from the bioreactor. If more sample volume is required an additional tube can be inserted; the skilled person will be aware that a variety of sample tube volumes are readily available, e.g. 2, 4, 5, 7, 10 or 15 mL. When sampling has been completed, clamp E is closed and the tubing is sealed, preferably in 3 locations, and then cut in the middle of the seals so that the sample can be aseptically separated from the sampling device. Thereafter claims B, C and D are opened and the plunger of the syringe (4) is pulled back and then pushed forward in order to purge the line. Clamps B, C and D are then closed. Further sampling instances may be carried out when required using the same procedure for another sub-conduit. In one embodiment the order of sampling is from right to left of FIG. 1, i.e. in the direction from the bioreactor end to the syringe end of the first conduit.

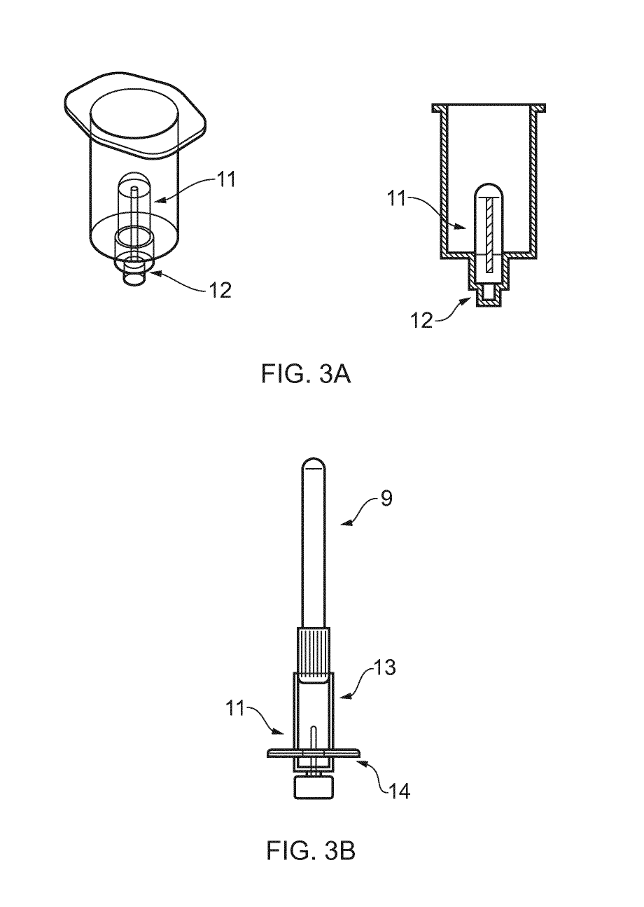

FIG. 3A shows a schematic view of a sleeved hollow needle with a Luer connection (12). This device may be inserted into a needless port, for example clave port, on the bioreactor. Alternatively this device may be positioned at a sub-port of a sub-conduit of the sampling device of the invention. The sleeved needle (11) is necessary to provide a connection between the bioreactor and the receiving vacuum tube.

FIG. 3B shows a schematic view of the complete access device comprising a protective housing (13) for receiving the vacuum tube (9) and having a needle (11) for penetrating the lid of the vacuum tube. The needle is provided with a protective sleeve of rubber or silicon. The access device is provided with a Luer or other connection for direct connection directly to a bioreactor or to a sub-port of the sampling device of the invention to enable sampling directly into the vacuum tube.

The following non-limiting example describes an embodiment of the present invention.

### EXAMPLE 1

### Cell Sampling of T-Cells Perfusion Cultured in Bioreactor

### Example 1(i): Cell Culture

Cryopreserved human peripheral blood mononuclear cells (PBMCs) were thawed, washed twice and cultured in T225 flasks at 1E06 cells per ml in X-VIVO™-10 (Lonza) supplemented with 5% heat-inactivated human serum (TCS), 2 mM GlutaMAX™ (Life Technologies), 1% penicillin/streptomycin (Life Technologies) and 20 ng/ml of IL-2 (Peprotech). Cell expander™ CD3/CD28 beads (Life Technologies) were added to the culture at a ratio of 3:1 beads: CD3+T cells. After 3 days incubation, cells were counted and maintained at 0.5E06 cells per ml for an additional 2 days.

On day 5 of the culture, the cells were transferred into a flexible 2 L bioreactor ( Xuri Cell bag Bioreactor, GE Healthcare) for culture on the WAVE Bioreactor 2/10 System and the Xuri Cell Expansion Systems W5 and W25 (GE Healthcare).

Once a minimal number of 5×108 cells were obtained in static culture, cells were transferred to 2 L Xuri Cell bag Biorectors with perfusion filter. Cell bags were loaded onto the WAVE Bioreactor 2/10 System, Xuri W5 and Xuri W25 Systems. All bioreactors were set at 37° C. with a rock rate of 10 rpm and a rock angle of 6°. Cells were maintained at 0.5×106 cells per ml and cultures fed batch until a maximum volume of 1000 mls was reached. Once the cell concentration had reached 2×106 cells/ml in the 1L culture, perfusion was commenced for the remainder of the expansion. Perfusion on the WAVE 2/10 and Xuri W5 Systems was run as semi-continuous perfusion with shot volumes of 50 mls. Continuous perfusion was used on the Xuri W25 system.

Example 1(ii): Cell Sampling

Cells were harvested once a resting state had been achieved. A daily cell sample was taken from the bioreactor by attaching the access device with sleeve protected needle (5) to the bioreactor via the connector (2). Vacuum tubes (4) were used to draw cell samples from the bioreactor.

The vacuum tubes are inserted onto the access device and the vacuum draws the cell suspension into the tube through the needle of the access device. The cell suspension can be transported to the next stage of the process or mixed with the contents of the tube for further analysis.

Example 1(iii): Phenotypic Analysis

The cells were immunophenotyped by flow cytometric analysis at days 0 and 10 of culture: 1E06 cells were stained with CD3-per CPCy5.5, CD4-PE, CD8-AlexaFluor488, CD28-APC and CD27-V450, or CD57-APC and CD62L-V450, and analysed on a FACS Fortessa flow cytometer using FACS Diva software, according to the manufacturer's instructions (reagents, instrument and software from BD Biosciences).

From the above it appears that the procedure to draw cell samples from bioreactors according to the invention is sterile and does not damage the sensitive cells. Furthermore the method is efficient which enables rapid and easy sample collection. If desired, the process may be automated.

1. A sampling device comprising:
a first conduit comprising a first port and a second port, wherein said first port is configured to be fluidly connected to a bioreactor and wherein said second port is fluidly connected to a venting device;
a plurality of sub-conduits having corresponding sub-ports, wherein each of the plurality of sub-conduits is fluidly connected to the first conduit at respective connector junctions, and wherein each of the sub-ports is fluidly connected to the first conduit and configured to be operatively coupled to a vacuum tube;
a plurality of flow controllers disposed along said first conduit between each of said connector junctions and disposed along each of said sub-conduits between said connector junction and said sub-port;
a plurality of vacuum tubes each configured to be operatively coupled to a respective sub-port of a corresponding sub-conduit.

2. The sampling device as defined in claim 1 wherein said venting device is a syringe.

3. The sampling device as defined in claim 1 wherein each of said plurality of flow controllers is a clamp.

4. The sampling device as defined in claim 1 wherein each sub-port comprises a needle-provided access device and wherein a fluid path is created through the needle of the access device.

5. The sampling device as defined in claim 1 wherein the vacuum tube is empty.

6. The sampling device as defined in claim 1 wherein vacuum tube contains a cell viability stain.

7. The sampling device as defined in claim 6 wherein the cell viability stain is taken from DRAQ5, Hoechst, or propidium iodide.

8. The sampling device as defined in claim 1 wherein vacuum tube contains single antibodies.

9. The sampling device as defined in claim 1 wherein vacuum tube contains multiple antibodies.

10. The sampling device as defined in claim 8 wherein vacuum tube contains anti-CD3, anti-CD4 or anti-CD8.

11. The sampling device as defined in claim 1 further comprising an air filter operatively coupled to the venting device.

12. The sampling device as defined in claim 1 wherein said first conduit is a continuous conduit.

13. A method comprising sampling cells from a bioreactor into a vacuum tube wherein said method comprises:
providing a sampling device as defined in claim 1 wherein said plurality of flow controllers are in a closed position;
aseptically connecting said sampling device to said bioreactor;
creating a closed fluid path from said bioreactor to a sub-port of said sampling device by opening a defined selection of said plurality flow controllers; and,
aseptically connecting said vacuum tube to said sub-port so that said cells to move from the bioreactor into said vacuum tube.

14. The method as defined in claim 13 wherein each sub-port comprises a needle-provided access device and wherein said fluid path runs through the needle of the access device.

15. The method as defined in claim 13 wherein said bioreactor is a flexible cell bag.

16. The method as defined in claim 13 wherein the container is empty.

17. The method as defined in claim 13 wherein container contains a cell viability stain.

18. The method as defined in claim 17 wherein the cell viability stain is taken from DRAQS, Hoechst, or propidium iodide.

19. The method as defined in claim 17 wherein the cells are instantly mixed with the viability stain; removing said container from said bioreactor, and analyzing for viable cell number.

20. The method as defined in claim 13 wherein container (9) contains single antibodies.

21. The method as defined in claim 13 wherein container contains multiple antibodies.

22. The method as defined in claim 20 wherein container contains anti-CD3, anti-CD4 or anti-CD8.

23. The method as defined in claim 20 comprising cultivating cells in said bioreactor, sampling cells from said bioreactor into said container provided with an antibody wherein the cells are instantly mixed with the antibody; removing said container from said bioreactor, and analyzing for antigen.
